(19) United States
(12) Patent Application Publication
(10) Pub. No.: US 2007/0293927 A1
(43) Pub. Date: Dec. 20, 2007

(54) Gene and Cell Delivery Self Expanding Polymer Stents

(75) Inventors: Ko Frank, Philadelphia, PA (US); Robert J. Levy, Herion Station, PA (US); Ivan Alferiev, Clementon, NJ (US); Ilia Fishbein, Philadelphia, PA (US)

Correspondence Address: RATNERPRESTIA, P O BOX 980, VALLEY FORGE, PA, 19482-0980, US

(73) Assignees: The Children's Hospital Of Philadelphia, Philadelphia, PA (US); Drexel University, Philadelphia, PA (US)

(21) Appl. No.: 10/589,711

(22) PCT Filed: Apr. 16, 2004
(86) PCT No.: PCT/US04/11794

§ 371 Date: Jun. 18, 2007

Related U.S. Application Data
(60) Provisional application No. 60/545,126, filed on Feb. 17, 2004.

(51) Int. Cl. A61F 2/06

(52) U.S. Cl. 623/1.11; 623/1.15

(57) ABSTRACT

A device having polymeric filaments, wherein at least one of the filaments includes at least one groove for slidably retaining at least one other filament, such that the device is adapted to revert to a tubular lattice structure when allowed to expand from a collapsed state. A device as described above and further including a biologically active function, wherein the polymeric filaments of the device include an agent having a reactive group or a fiber adapted to covalently react with a biomaterial. Thus, the device of the invention has an active structural function such as the ability to regain a shape and, optionally, a biologically active function such as the ability to deliver a biomaterial to an organism or a cell. A process of manufacturing the device is also described.

### CROSS-REFERENCE TO RELATED APPLICATIONS

This application claims the benefit of provisional Application No. 60/545,126, filed Feb. 17, 2004, titled GENE AND CELL DELIVERY SELF EXPANDING POLYMER STENTS which is incorporated herein in its entirety.

### STATEMENT REGARDING FEDERALLY SPONSORED RESEARCH OR DEVELOPMENT

This research was supported in part by U.S. Government funds (National Heart Lung and Blood Institute grant numbers HL59730 and HL72108), and the U.S. Government may therefore have certain rights in the invention.

### BACKGROUND OF THE INVENTION

1. Field of Invention

This invention relates to expandable vascular repair devices, endoprosthesis devices or stents for implanting in a body lumen.

2. Description of Related Art

Stents are implantable devices used in a body's lumen to maintain the patency thereof. The stent delivery system is useful in the treatment and repair of body lumens, including coronary arteries, renal arteries, carotid arteries, and other body lumens.

Stents are generally cylindrically-shaped devices which function to hold open and sometimes expand a segment of a blood vessel or other body lumen. They are particularly suitable for supporting and holding back a dissected arterial lining which can occlude the fluid passageway. Further, stents are useful in maintaining the patency of a body lumen, such as a coronary artery, after a percutaneous transluminal coronary angioplasty (PTCA) procedure or an atherectomy procedure to open a stenosed area of the artery.

A variety of devices are known in the art for use as stents and have included coiled wires in a variety of patterns that are expanded after being placed intraluminally by a balloon catheter; helically wound coil springs manufactured from an expandable heat sensitive material such as nickel-titanium; and self-expanding stents inserted in a compressed state and shaped in a zig-zag pattern.

Stents have traditionally been used primarily for structural functions as a means for minimally invasive treatment of aneurysms and atherosclerosis. Although stents have been known to be in existence since the 1700's, modern use of stents did not gain popularity until the 1980's with the development of the Wall stent (see Mueller, R. and Sanborn, T., The History of Interventional Cardiology, Am. Heart J, 1995; 129:146-172).

The balloon expandable stents such as the Palmaz-Schatz stents is an example of FDA approved stents currently being used (see King, S. B., Angioplasty from bench to Bedside to Bench, Circulation, 1996, 93:1621-1629). These stents are made of metals such as stainless steel (Palmaz-Schatz and Wall), tantalum (Cordis, Strecker, Wiktor, Mayo), and Nitinol (SciMed, Angiomed-USCI, Cardiocoil). Although the metallic stents have sufficient mechanical properties and radiopacity, they tend to be too stiff for the blood vessels. The rigidity of metallic stents not only makes them difficult to deploy into specific sites but also poses a threat of rupturing the blood vessel during deployment. When bending over sharp curvatures, metallic stents tend to stretch beyond their elastic limit, undergo plastic permanent deformation, and therefore prevent the stent from proper recovery to its intended geometry. Over a prolong period of deployment, metals tend to fatigue and cause deterioration of radial strength and loss of their intended function. Metallic stents are also known to occlude prematurely after deployment and case restenosis (see Ahmed M, Bishop, M. C., Bates, C. P., Mmanhire, A. R., Metal Mesh stents for ureteral obstruction by hormone-resistant carcinoma of postate, J. Endourol, 1999, April, 13(3): 221-224).

Further details of prior art stents can be found in U.S. Pat. Nos. 6,432,133, 6,596,022, 6,511,504, 6,626,933, and 6,629,991 to Lau et al., 6,635,084 to Israel et al., 6,576,006 to Limon et al, and 6,629,994 to Gomez et al.

Considering the deficiencies of metallic stents, there is a need for material systems and structures that are flexible, conformable, easily deployable, and biocompatible with tailorable strength. With the availability of a large family of polymers, there are increasingly more effective design options to address the deficiencies of metallic stents by using polymeric stents.

One of the problems associated with the prior art stents relates to recovery of structural dimensions of stents. In order to deliver the stent to the body lumen, it is deformed, compressed or crimped onto a delivery device such as catheter. Once the stent is deployed, it is expected to recover its original shape. Many attempts have been made to address the issue of maintaining the shape of a stent inside the lumen. One of the ways to maintain the shape is by manufacturing the stent using a shape memory material as described in U.S. Pat. No. 6,635,079 to Unsworth et al., wherein the shape of a device made from the shape memory material is set by heating the material at certain high temperatures. The shape can then be deformed at lower temperatures and recovered by heating to high temperatures.

Along with a structural function, stents have been used for preventing and treating diseases by delivering drugs and cells to targeted areas of a body. For example, U.S. Pat. No. 6,613,084 to Yang discloses a stent with drug delivery capabilities, and U.S. Pat. No. 6,599,274 to Kucharczyk, et al. discloses a cell delivery device.

Moreover, stents by themselves can cause problems such as local trombosis. To combat these problems, patients require administering treatment with anti-coagulant and antiplatelet drugs. U.S. Pat. No. 6,613,084 to Yang discloses delivery of such drugs associated with a cover attached to a stent.

Despite the foregoing developments, there is still a need in the art for alternative stent designs.

All references cited herein are incorporated herein by reference in their entireties.

### BRIEF SUMMARY OF THE INVENTION

The invention makes use of the intertwined nature of braided polymeric structures wherein the braiding yarns (mono and/or multifilaments) render a multitude of functions. These functions include a structural function and a bioactive function such as, for example, a gene and cell delivery function.

Accordingly, the invention provides a device comprising polymeric filaments, wherein at least one of the filaments includes at least one groove for slidably retaining at least one other filament, such that the device is adapted to revert to a tubular lattice structure when allowed to expand from a collapsed state.

In certain embodiments, the tubular lattice structure has an expanded transverse diameter substantially identical to a manufactured transverse diameter. Preferably, the manufactured transverse diameter is about 2 mm to about 300 mm.

In certain embodiments, the manufactured transverse diameter is heat set at a temperature equal to or above a glass transition temperature of the filaments. Preferably, the temperature is 100-200° C.

In certain embodiments, the filaments have a diameter from about 10 micron to about 1000 micron.

In certain embodiments, at least three of the polymeric filaments are braided at a braiding angle of about 5° to about 85°. Preferably, the braiding angle is from 40° to 50°.

In certain embodiments, at least one groove has a combination of depth and height suitable to inhibit a movement of adjacent filaments in a transverse direction beyond the manufactured transverse diameter without inhibiting the movement in a longitudinal direction.

In certain embodiments, at least one of said filaments further comprises a plurality of grooves.

In certain embodiments, the polymeric filaments comprise a thermoplastic polymer, wherein the polymer is selected from a group consisting of poly(ester), poly(lactic acid), poly(glycolic acid), poly(lactide-co-glycolide), poly(caprolactone), mixtures and copolymers thereof.

In certain embodiments, the polymeric filaments are at least one of monofilaments or multifilaments.

In certain embodiments, the tubular lattice structure as defined above is a stent.

Also provided is a device comprising polymeric filaments, wherein at least one of the filaments includes at least one groove for slidably retaining at least one other filament, such that said device is adapted to expand from a collapsed state to form a tubular lattice structure having an expanded transverse diameter substantially identical to a manufactured transverse diameter.

Further provided is a process of manufacturing the device, the process comprising:

providing polymeric filaments;

arranging the polymeric filaments over a mandrel to form a tubular lattice assembly having interlacing junctions, wherein the mandrel has the manufactured transverse diameter;

heating the tubular lattice assembly at a temperature of at least 10° above a glass transition temperature of the polymeric filaments; and

indenting at least one of the polymeric filaments at one or more of the interlacing junctions to make at least one groove on at least one of the polymeric filaments for slidably retaining at least one other polymeric filament and thereby forming the tubular lattice structure.

In certain embodiments, the process further comprises contacting at least one of the filaments with an agent having a reactive group, wherein the reactive group is adapted to covalently react with a biomaterial.

In certain embodiments, the process further comprises covalently attaching the agent to at least one of the filaments.

In certain embodiments, the process further comprises contacting at least one of the filaments with a fiber associated with the agent. In one variant of the process, the fiber has a diameter of about 5 nm to about 10 microns.

In certain embodiments of the process, contacting is done by at least one of an ultrasonic welding or an electrospinning process.

In certain embodiments, the process further comprises covalently attaching the agent to at least one of the filaments.

Further provided are a stent delivery system and a method of manufacturing thereof.

Also provided is a method for delivery of a biomaterial to a cell using the device of the invention.

### BRIEF DESCRIPTION OF SEVERAL VIEWS OF THE DRAWINGS

The invention will be described in conjunction with the following drawings in which like reference numerals designate like elements and wherein:

FIG. 1 is a schematic illustration of triaxial braiding.

FIG. 2 is a schematic illustration of structural hierarchy of braided structures.

FIG. 3 is a schematic illustration of a structure of a triaxial braid.

FIG. 4 is scheme demonstrating a braid formation over a mandrel.

FIG. 5 is graph illustrating a processing model of a braided stent, wherein a fiber volume fraction (V_{f}) and a braid angle (Θ) vary with change in a braid tightness factor (η).

FIG. 6A is a schematic illustration of a braided structure having various filaments. The filaments are shown in FIGS. 6B, 6C, and 6D wherein FIG. 6B is a filament with nanofibers 18, FIG. 6C is a filament with textures 20 having different firmness, and FIG. 6D demonstrates the filament before it is functionalized with the fiber or textures.

FIGS. 7A and 7B are images showing a stent in a stretched state and in a deployed state respectively.

FIG. 8 is a scheme illustrating a process of attachment of fibers to a monofilament by an ultrasonic welding (prior art).

FIGS. 9A and 9B are images showing an attachment of fibers to a rotating braided stent by electrospinning.

FIG. 10 is a scheme illustrating a stent manufacturing process including braiding of filaments to make a tubular lattice structure, heat setting of under pressure in a heated die wherein grooves are made on filaments, and post-curing in the oven. Next, the stents are pulled through the die before being cut to a different length.

FIG. 11 is a scheme depicting synthesis of water-soluble photo-activatable polymer for use as an agent on stents for attaching biomaterial.

### DETAILED DESCRIPTION OF THE INVENTION

The invention was driven by a desire to develop a device capable of retaining its shape upon deployment and release from a deformed state and optionally capable of delivery of a biomaterial such as, for example, genes and cells to an organism or a cell. Thus, the device of the invention has an active structural function such as the ability to regain a shape through a programmed self-expansion mechanism and, optionally, a biologically active function such as the ability to deliver a biomaterial to an organism or a cell.

Accordingly, the invention provides a device comprising polymeric filaments, wherein at least one of the filaments includes at least one groove for slidably retaining at least one other filament, such that the device is adapted to revert to a tubular lattice structure when allowed to expand from a collapsed state.

In certain embodiments of the invention, the device is a self-expandable polymeric stent (SEPS). The device of the invention can be used for patients with cancer causing obstructive manifestations as well as for other applications listed above. When the device of the invention is functionalized with a biomaterial, it can be used for administering therapies for various diseases such as, for example, cystic fibrosis and stenosis.

The device possesses a structurally active function, wherein it comprises monofilaments and/or multifilaments made of thermoplastic polymers; monofilaments and/or multifilaments are braided and subsequently thermally programmed to retain a geometric shape, orientation and dimensions by heat setting and compression.

Further, in certain embodiments of the invention, the device possesses a biologically active function, wherein monofilaments and/or multifilaments of the device are coupled with a biomaterial and/or with an agent having a reactive group adapted to react with a biomaterial.

The agent is coupled with polymeric filaments of the device by methods known in the art, for example, by photo-activation or by chemical modification.

Design and Fabrication of Device of the Invention

Key features in the design and fabrication of the device of the invention include:

1. Geometric and material design of polymeric filaments;

2. Extrusion of polymeric filaments;

3. Designing of a braided structure by multi-scale hierarchical engineering and manufacturing; and

4. Programmed self-expansion mechanism or a shape memory design based on heat setting and proper compression to achieve various degree of self-locking; and

5. Conferring a bioactive function by functionalizing polymeric filaments with an agent having a reactive group and/or a biomaterial.

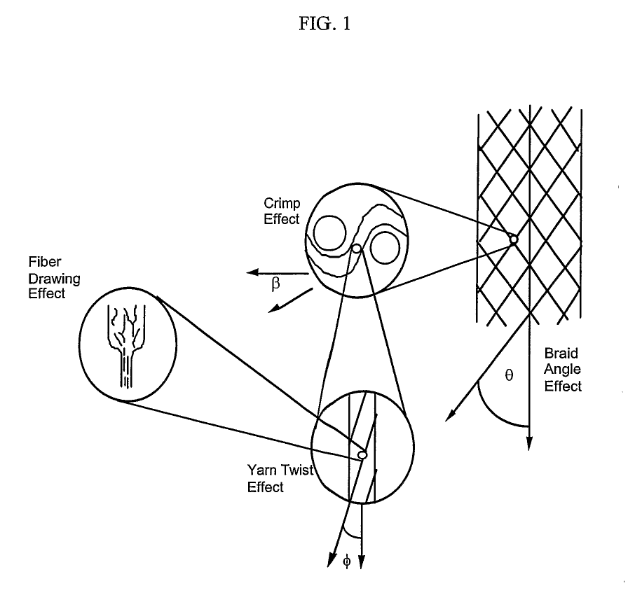

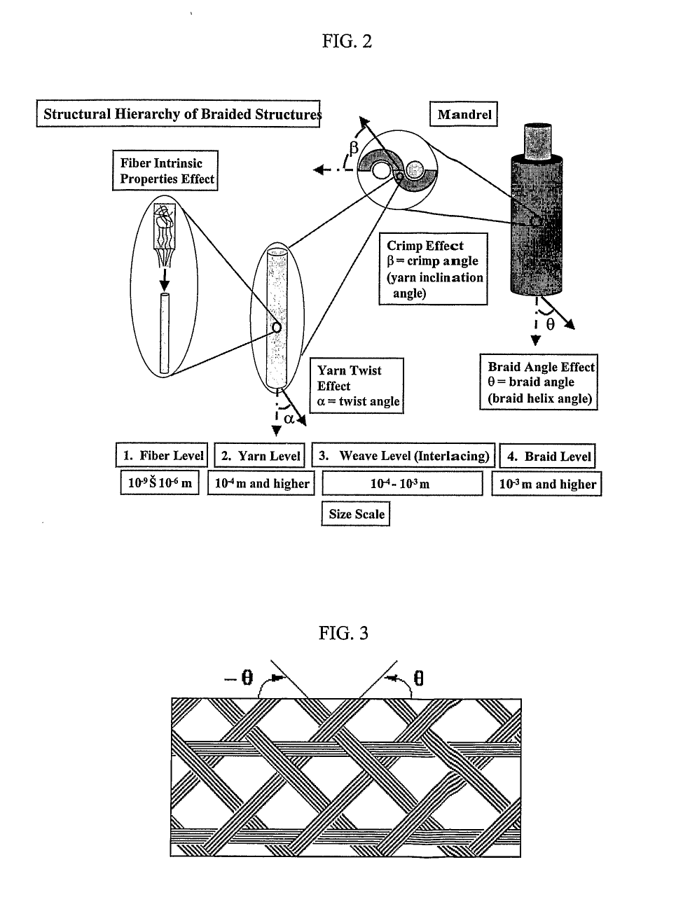

The design concept for the braided self-expandable polymeric stent (SEPS) is based on the approach of integrated design for manufacturing (IDFM), wherein systematic tracking of properties translation from a single filament to the final braided structure is carried out. (Naidu, N. S, Self-Expanding Polymeric Stents, Drexel University M.S. Thesis, 2001). This fiber architecture-based hierarchical design methodology is illustrated in FIGS. 1 and 2. There are four levels of textile integration from fiber to the braided tubular structure: 1) a fiber level, where molecular structures and bonds dominate the properties; 2) the yarn level, wherein the monofilament or multifilament yarn is formed by extrusion and/or spinning to create twisted and textured structures and wherein the translation in properties from fiber to yarn is affected by the fiber helix angle (μ); 3) the weave level, including the crimp effect (the crimp angle (β)) from interlacing of the filaments at crossovers; and 4) the braid level, wherein the orientation (the braid angle (θ)) of braiding yarns (a monofilament, a multifilament or a combination of both) relative to the fabric's axis is the affecting factor. Systematic tracking of the translation of properties at each level of integration allows accurate prediction of composite stress-strain properties. By proper selection of the braiding parameters, the geometry and dimensions of the braided stent can be designed and manufactured to desired specification (see Ko et al., Handbook of Industrial Braids, 1989, and Ko, F. K. “Braiding” in ASM Handbook, Vol. 21, Composites, ASM International, December, 2001, pp. 69-77). The design concept is generic, and thus, it is applicable to any braided tubular structures. Accordingly, a family of self-expandable braided stents can be produced by materials' hybridization, fiber architecture design, surface texture design and proper selection of braiding parameters, post processing heat setting and pressuring techniques.

The implementation of the design concept can be carried out at various level of processing technology. At the fiber/yarn spinning level (a yarn is a linear assembly of fibers), the polymers (e.g., polyurethane, polyester) used in this invention with and without biomaterial are extruded in monofilament and multifilament form.

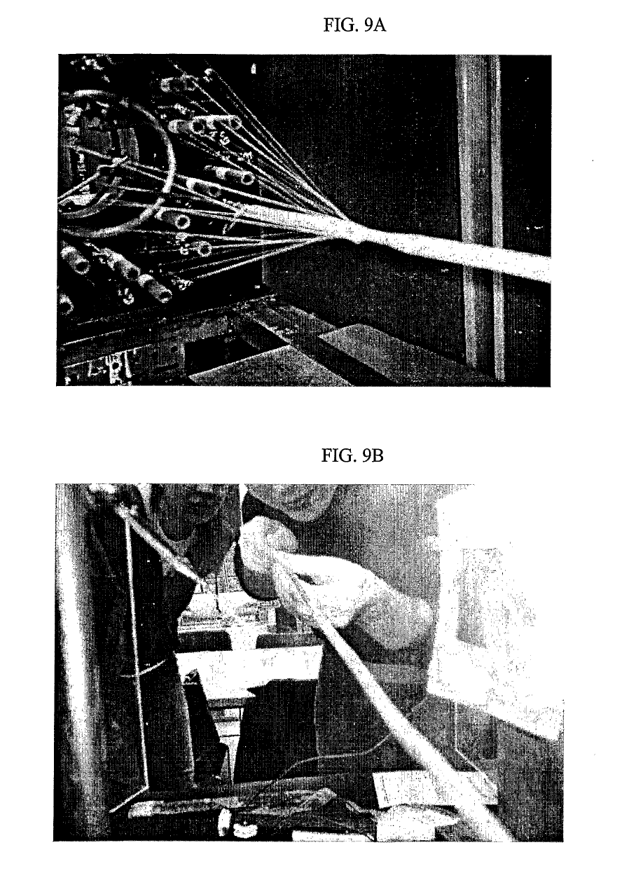

To create a more uniform dispersion of the biomaterial, nanoscale fibrils containing a mixture of the polymer/biomaterial are co-spun using the electrospinning process (Fertala, A., Han, W. B. and Ko, F. K., “Mapping Critical Sites in Collagen II for Rational Design of Gene-Engineered Proteins for Cell-Supporting Materials,” J. Biomed Mater Res 57, 48-51, 2001, Li, W.-J., Laurencin, C. T., Caterson, E. J, Tuan, R. S, and Ko, F. K., “Electrospun Nanofibrous Structure: A Novel Scaffold for Bioengineering,” Journal of Biomedical Materials Research, Wiley Interscience, Mar. 25, 2002, pp. 613-621) as shown in FIG. 9.

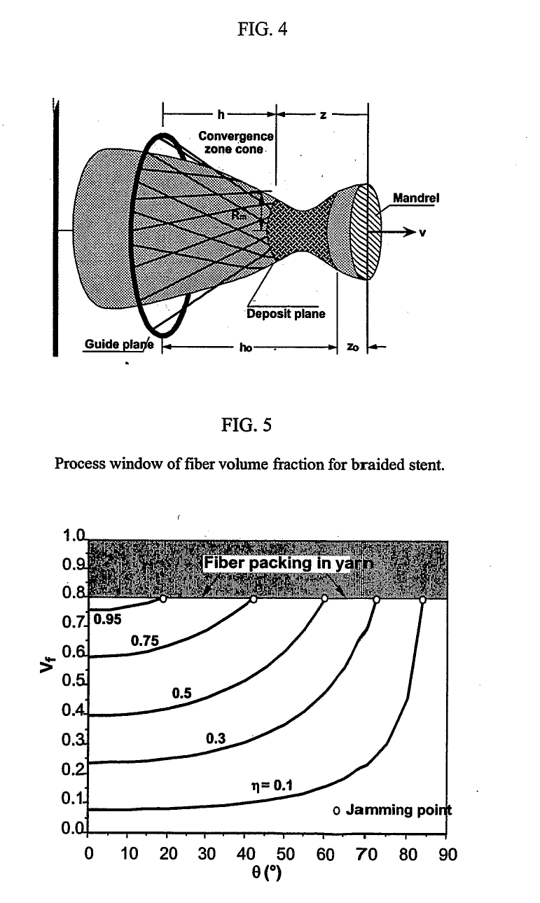

The monofilament and or the nanoscale hybrid fibrous structures are then co-braided as shown in FIG. 4 to create a tubular lattice structure (a braided structure). Based on the deployed dimension specified for the stent, braiding is carried over a mandrel of an appropriate diameter.

Next, shape memory design is implemented by (1) heat setting of the braided assembly at an appropriate temperature depending on the polymer used and (2) compression to achieve various degree of self-locking. Preferably, both actions are done simultaneously.

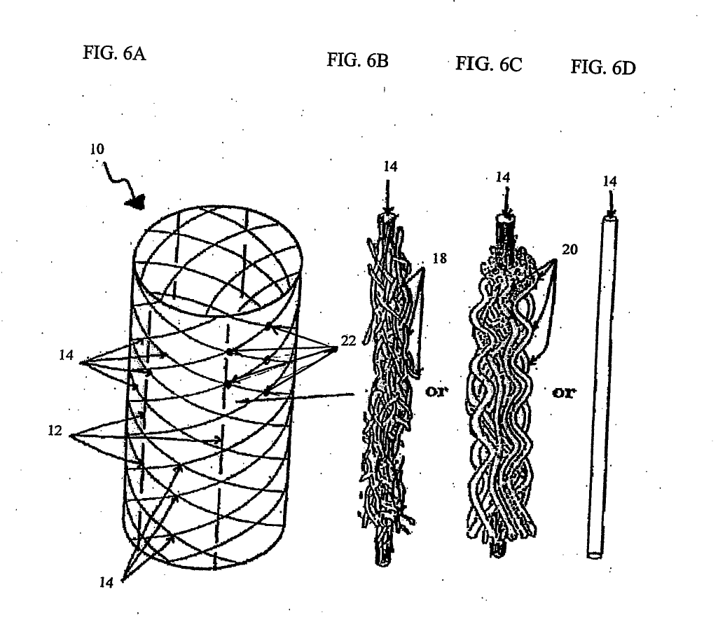

Braided structures (e.g., fabrics) can be produced in a tubular form or a tubular lattice structure by intertwining three or more yarns together. The bias interlacing nature of the braided fabrics makes them highly conformable. Triaxial braiding can be produced by introducing 0° yarns 12 as shown in FIG. 6 to enhance reinforcement in the 0° direction. The fiber type and the braiding angle can be varied as needed.

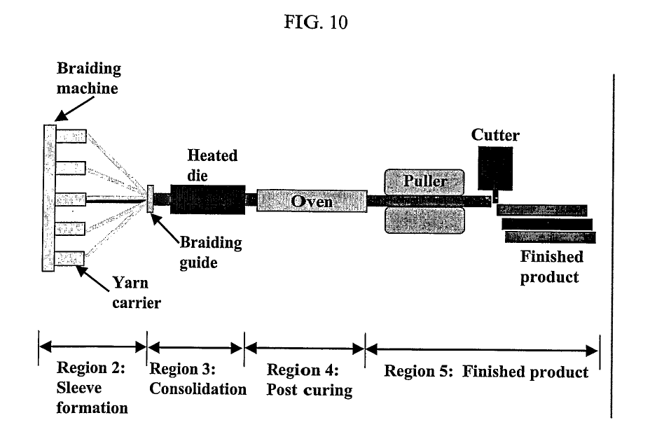

FIG. 6A demonstrates the tubular lattice structure 10, made by braiding filaments 14. After braiding, filaments 14 are treated in a heated die as shown in FIG. 10 to receive grooves 22.

FIGS. 6B and 6C demonstrate various types of functionalized filaments 14 such as a filament functionalized with nanofibers 18 or textures 20 having different firmness and a filament functionalized with an agent for attaching biomaterials (not shown). FIG. 6D demonstrates the filament before it is functionalized with the fiber or textures. At least one of the filaments 14 can be functionalized by attaching a biomaterial or an agent capable of reacting with a biomaterial. Optionally, the tubular lattice structure 10 can be fortified by addition of one or more “lay-in” filaments (0° yarns) 12, which may also by functionalized to carry a bioactive function as described above.

The formation of shape and fiber architecture of the tubular lattice structure is illustrated in FIGS. 3 and 4. FIG. 4 depicts the process of braiding over an axisymmetric or a symmetric shape of revolution according to instructions generated through the process kinematic model. Governing equations for this processing model and input parameters form the basis for a computer controlled braiding process (see Ko et al., supra, and Ko, F. K. supra).

TABLE 1
Key Input/Output

| | | | |
|---|---|---|---|
| Inputs: | | Key inputs/outputs: | |
| Constants | | Local braid angle | θ(z) |
| Guide radius | R_{g} | Local yarn volume fraction | V_{y}(z) |
| Number of carriers | N_{c} | Machine speed profiles | v(t), ω(t) |
| Yarn width | w_{y} | Auxiliary outputs: | |
| Mandrel shape | Rₘ(z) | Convergence zone length | h(t) |
| Initial Conditions | | Local cone half-angle | γ(z) |
| Convergence zone length | hₒ | | |
| Starting deposit location | zₒ | Velocity of braid formation | $\frac{dz ( t )}{dt}$ |

TABLE 2
Governing Equations

| Convergence length | |
|---|---|
| $v ( t ) = \frac{dh ( t )}{dt} + \frac{{R}_{m} ( z ) ⊇ ω ( t ) ⊇ h ( t )}{{R}_{g} \sqrt{1 - {[ \frac{{R}_{m} ( z )}{{R}_{g}} + \frac{h ( t )}{{R}_{g}} tan γ ( z ) ]}^{2}}}$ | (6.5.4-1) |
| Braid angle | |
| $θ ( z ) = {tan}^{- 1} \{ \frac{{R}_{g}}{h ( t )} cos γ ( z ) \sqrt{1 - {[ \frac{{R}_{m} ( z )}{{R}_{g}} + \frac{h ( t )}{{R}_{g}} tan γ ( z ) ]}^{2}} \}$ | (6.5.4-2) |
| Fiber volume fraction | |
| ${V}_{f} ( z ) = \frac{κ ⊇ {w}_{y} ⊇ sin γ ( z )}{2 ⊇ {R}_{m} ( z ) ⊇ cos θ ( z ) ⊇ sin [ \frac{2 ⊇ π ⊇ sin γ ( z )}{{N}_{c}} ]}$ | (6.5.4-3) |
| Yarn jamming criterion | |
| ${θ}_{max} ( z ) = {cos}^{- 1} \{ \frac{{w}_{y} ⊇ sin γ ( z )}{2 ⊇ {R}_{m} ⊇ sin [ \frac{2 ⊇ π ⊇ sin γ ( z )}{{N}_{c}} ]} \}$ | (6.5.44) |

Geometric parameters include distributions of braiding angles, yarn volume fraction, and fabric covering factor (V_{f}) along the mandrel length. Processing variables include profiles of the braiding and mandrel advance speeds versus processing time. Equations (6.5.4-1)-(6.5.4-6) define the relationship between geometric parameters and processing variables, describe current machine status (braid length and convergence length), and provide process limits due to yarn jamming.

Braiding angle can range from 5° in almost parallel yarn braid to approximately 85° in a “hoop” yarn braid. However, because of geometric limitations of yarn jamming, the braiding angle that can be achieved for a particular braided fabric, as defined by Equation (6.5.4.6), depends on the following parameters: number of carriers N_{c}, braiding yarn width w_{y}, mandrel radius Rₘ, and half cone angle γ of the mandrel.

When the mandrel has a cylindrical shape, i.e., γ=0, the fiber volume fraction of the biaxial braid becomes:

$${V}_{f} = \frac{κ ⊇ {w}_{y} ⊇ {N}_{c}}{4 ⊇ π ⊇ {R}_{m} ⊇ cos θ} \qquad ( 6.5 .4 \text{-} 5 )$$

where κ is the fiber packing fraction, w_{y} is the yarn width, N_{c} is the number of braiding carriers, Rₘ is the radius of mandrel, and θ is the orientation angle of yarns. If the braid tightness factor η is defined as the ratio of the total width of either +θ or −θ yarns to the mandrel perimeter, as shown by the equation (6.5.4-6) below:

$$η = \frac{{w}_{y} ⊇ {N}_{c}}{4 ⊇ π ⊇ {R}_{m}} ( 0 < η \leq 1 ) \qquad ( 6.5 .4 \text{-} 6 )$$

the braid tightness factor must be maintained within the range of 0 to 1 to avoid yarn jamming. Combining Equations (6.5.4-5) and (6.5.4-6), the fiber volume fraction is expressed as shown in the equation (6.5.4-7) below:

$${V}_{f} = κ \frac{η}{cos θ} \qquad ( 6.5 .4 \text{-} 7 )$$

FIG. 5 shows the process window for the fiber volume fraction versus the braid angle at various levels of fabric tightness factor based on Equation (6.5.4-3). The fiber packing fraction again is assumed to be 0.8. Thus, for a given fabric tightness factor, the fiber volume fraction can be controlled by varying the braid angle, until the yarn jamming point is reached. In designing the braided stent, the fiber volume fraction (coverage) and fiber orientation angles are determined based on the desired recovery power, the hoop strength and the level of the biomaterial carrying capacity required. To achieve the requirement for the desired fiber volume fraction and orientation angle, it is only necessary to select a specific fabric tightness factor (either by changing the braiding carrier numbers, the width of braiding yarns, or a combination of the two) as defined by Equation (6.5.4-3).

### Shape Memory Design

Programmed self-expansion mechanism or a shape memory design is effectuated by heat setting and proper compression to achieve various degree of self-locking. Guided by the processing model as graphically illustrated in FIG. 5, the braided stent with the desirable braiding angle and coverage is placed on a mandrel of appropriate diameter for subsequent heat setting at a temperature on or above the glass transition temperature (T_{g}). Sufficient level of pressure from about 0.1 MPa to about 2 MPa is applied to the interlacing points of the braided stent such that mutual locking impression points are formed. The pressure can be applied, for example, by using a heated mold (e.g., a heated die).

Mutual locking impression points can also be described as grooves formed on polymeric filaments under the pressure. The function of grooves on one filament is to slidably retain at least one other filament, such that the stent is adapted to revert to a tubular lattice structure when allowed to expand from a collapsed state. Each filament has at least one groove; preferably, each filament has multiple grooves; even more preferably each place of contact of two or more filaments has a groove associated with it as shown in FIG. 6A.

Dimensions of each groove depend on the dimensions of filaments. For example, a groove has a combination of a depth and a height suitable to inhibit a movement of adjacent filaments in a transverse direction beyond the manufactured transverse diameter without inhibiting the movement in a longitudinal direction.

The term “manufactured transverse diameter” means the diameter of the mandrel. In certain embodiments, the manufactured transverse diameter is about 2 mm to about 300 mm. The diameter of the mandrel is also the deployed diameter of the stent, and it is selected in such a way that the deployed stent will have sufficient traction on the arterial wall.

Polymeric filaments preferably comprise a thermoplastic polymer. Non-limiting examples of the thermoplastic polymer include polyester, polyurethane, polylactic acid, polyglycolic acid, polylactide-co-glycolide, polycaprolactone, mixtures and copolymers thereof. Polymeric filaments can be biodegradable or non-biodegradable.

The purpose of the heat-setting step is to set the geometry (which includes diameter and filament orientation) of the tubular lattice structure. The heat step is performed at temperatures at or above T_{g} of polymeric filaments. In certain embodiments, the heat-setting step is performed at about 100° C. to about 200° C. and the heating is conducted for at least 30 minutes. Preferably, the temperature is from 100° C. to 150° C. In certain embodiments, the heat-setting step is performed at 200° C.

After heat setting in a heated die, the braided stent on the mandrel is cooled down to a room temperature, optionally after a post-curing step in an oven.

Next, the heat set tubular lattice structure (e.g., a double spiral of the braided stent) is stretched and straightened to its jamming point to fit into a catheter or another suitable delivery device. Upon deployment (release) from the catheter, the heat set diameter is recovered, thus anchoring the device on the designated location and supporting the arterial wall. In case of devices having the biomaterial coupled with filaments, the biomaterial can be delivered upon deployment of the device.

### Material Systems for Structural and Bioactive Functions

The braiding yarn can be in a form of a monofilament, a nanofiber wrapped monofilament or a textured filament as shown in FIGS. 6B and 6C respectively.

Monofilaments are preferably made of polymers, which can be combined in various hybridized blend of materials and geometric forms. Monofilaments and/or fibers can be made from thermoplastic, non-thermoplastic, and organometallic polymers. In certain embodiments of the invention, the polymeric filaments comprise thermoplastic polymers. Non-limiting examples of thermoplastic polymer are poly(ester), poly(urethane), poly(lactic acid), poly(glycolic acid), poly(lactide-co-glycolide), poly(caprolactone), mixtures and copolymers thereof.

Fibers used in this invention have a diameter of about 5 nm to about 10 microns. Fibers can be made and deposited on filaments by electrospinning process, spraying, ultrasonic welding or other methods known in the art. Fibers can be made from poly(vinylidenefluoride) (PVDF), poly(vinylidene fluoride-co-hexafluoropropylene), poly(acrylonitrile), poly(acrylonitrile-co-methacrylate), poly(methylmethacrylate), poly(vinylchloride), poly(vinylidenechloride-co-acrylate), poly(ethylene), poly(propylene), nylons such as nylon-12 and nylon-4,6, aramid, poly(benzimidazole), poly(vinylalcohol), cellulose, cellulose acetate, cellulose acetate butylate, poly(vinyl pyrrolidone-vinyl acetates), poly(bis-(2-methoxy-ethoxyethoxy))phosphazene(MEEP), poly(ethylene imide) (PEI), poly(ethylene succinate), poly(ethylene sulphide), poly(oxymethylene-oligo-oxyethylene), poly(propyleneoxide), poly(vinyl acetate), polyaniline, poly(ethylene terephthalate), poly(hydroxy butyrate), poly(ethylene oxide), SBS copolymer, poly(lactic acid), polypeptide, biopolymer such as protein (e.g. silk), pitch series such as coal-tar pitch and petroleum pitch, electrically conductive polymers such as poly(ethylenedioxithiothene) and poly(aniline) and their blends. Copolymers and blends of the above polymers may be used. Also, it is possible to use blends in which emulsions or organic or inorganic powders are blended with the above polymers. Aramid fibre is a man-made organic polymer (an aromatic polyamide) produced by spinning a solid fibre from a liquid chemical blend. It is also known under various trade names such as KEVLAR by DuPont (Willmington, Del.) and TWARON by Akzo Nobel Inc (Chicago, Ill.).

When nanofibers are applied onto a filament, they form a coating on the surface of the filament.

The term “coating”, as used herein, includes coatings that completely cover a surface, or a portion thereof (e.g., continuous coatings, including those that form films on the surface), as well as coatings that may only partially cover a surface, such as those coatings that after drying leave gaps in coverage on a surface (e.g., discontinuous coatings). The later category of coatings may include but is not limited to a network of covered and uncovered portions and distributions of nanofibers on a surface, which may have spaces between the nanofibers. In some embodiments, the coating preferably forms at least one layer of nanofibers on the surface, which has been coated, and is substantially uniform. However, when the coatings described herein are described as being applied to a surface, it is understood that the coatings need not be applied to, or that they cover the entire surface. For instance, the coatings will be considered as being applied to a surface even if they are only applied to modify a portion of the surface.

In certain embodiments, the device of the invention possesses a bioactive function. Bioactive function can be conferred to the device by associating filaments and/or fibers with an agent and/or a biomaterial. In certain embodiments, the polymeric filaments are associated with an agent having a reactive group adapted to covalently react with a biomaterial. In certain embodiments, polymeric filaments are associated with a fiber that is in communication with the agent and/or the biomaterial. For example, the agent and/or the biomaterial can be applied to the fiber by coating, painting, stamping, printing, and/or spraying. In another variant, the agent and/or the biomaterial are covalently attached to the fiber. In yet another variant, the fiber is made essentially of the agent and/or the biomaterial.

Agent

The agent used in the present invention is a polymer comprising a reactive group, wherein the reactive group is adapted to covalently react with a biomaterial. The preferred agent used in the invention is described in a co-pending PCT application titled MAGNETICALLY CONTROLLABLE DRUG AND GENE DELIVERY STENTS by Levy et al filed on even day therewith.

Non-limiting examples of the polymer used to make the agent include polymers comprising at least one monomer selected from the group consisting of allylamine, vinylamine, acrylic acid, carboxylic acid, alcohol, ethylene oxide, and acyl hydrazine. Preferably, the polymer of the agent is poly(allylamine).

The agent can be prepared by using methods known in the art from a polymer (biodegradable or non-biodegradable) comprising reactive groups and hydrophilic groups, which is then modified to contain photo-activatable groups. Also, it can be prepared by polymerization of monomeric blocks containing the above groups. In certain embodiments of the invention, the poly(allylamine) has a molecular weight of about 200 KDa to about 5 KDa. In the preferred embodiment, the molecular weight is from 70 KDa to 15 KDa.

In certain embodiments, the reactive group is a member selected from the group consisting of an amino group, a thiol-reactive group, a carboxy group, a thiol group, a protected thiol group, an acyl hydrazine group, an epoxy group, an aldehyde group, and a hydroxy group.

In certain embodiments, the thiol-reactive group is a member selected from the group consisting of a 2-pyridyldithio group, a 3-carboxy-4-nitrophenyldithio group, a maleimide group, an iodoacetamide group, and a vinylsulfonyl.

In certain embodiments, the agent is covalently attached to the at least one of the filaments.

In certain embodiments, the agent is a water-soluble photo-activatable polymer comprising: (a) a photo-activatable group, wherein the photo-activatable group is adapted to be activated by an irradiation source and to form a covalent bond between the water-soluble photo-activatable polymer and a surface having at least one carbon, (b) a reactive group, wherein the reactive group is adapted to covalently react with the biomaterial, (c) a hydrophilic group, wherein the hydrophilic group is present in an amount sufficient to make the water-soluble photo-activatable polymer soluble in water and (d) a polymer precursor.

In certain embodiments, the water-soluble polymer is photo-active polyallylamine benzophenone (PAA-BzPh) represented by a formula: 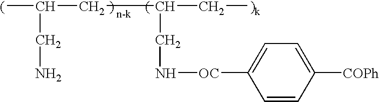
wherein n is 50 to 2000 and k is 10 to 1000.

In certain embodiments, the water-soluble polymer is polyallylamine based benzophenone further modified to contain 2-pyridyldithio groups (PDT-BzPh) represented by a formula:

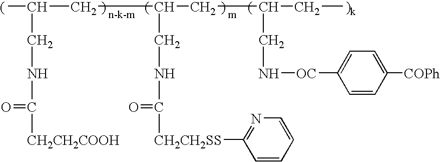

wherein n is 50 to 2000, k is 10 to 1000, and m is 10 to 1000.

The term “photo-activatable group” used herein denotes chemical groups capable of generating active species such as free radicals, nitrenes, carbenes and excited states of ketons upon absorption of external electromagnetic or kinetic (thermal) energy. These groups may be chosen to be responsive to various portions of the electromagnetic spectrum, i.e., the groups responsive to ultraviolet, visible and infrared portions of the spectrum. The preferred photo-activatable groups of the invention are benzophenones, acetophenones and aryl azides. Upon excitation, photo-activatable groups are capable of covalent attachment to surfaces comprising at least one carbon such as polymers.

The water-soluble photo-activatable polymer may have one or more photo-activatable groups. In certain embodiments, the water-soluble photo-activatable polymers have at least one photo-activatable group per molecule. Preferably, the water-soluble photo-activatable polymers have a plurality of photo-activatable groups per molecule. More preferably, photo-activatable groups modify at least 0.1% of monomeric units of a polymer precursor, even more preferably at least 1%, and most preferably from about 20 to about 50%.

The agent can be attached to filaments prior to or after braiding by methods known in the art. If the agent has photo-active groups, the attachment is done by irradiation and thereby forming the monomolecular layer of the agent. The irradiation source can be any source known in the art capable of emitting the light having a wavelength absorbable by the photo-activatable group of the invention. A UV-lamp is preferred when the benzophenone is used as the photo-activatable group. In certain embodiments of the method of making the device of the invention, the irradiation is performed at a wavelength from about 190 to about 900 nm. Preferably, the irradiation is performed at a wavelength of 280 to 360 nm.

Biomaterial

The biomaterial used in the present invention can be any molecule or macromolecule, which has a therapeutic utility such as for example gene therapy or it can be a prophylactic agent useful in the prevention of disease. Preferably, the biomaterial is any molecule or macromolecule to which a suitable reactive group, such as a carboxy (—COOH), amino (—NH₂) or thiol group (—SH) is attached. For example, proteins or peptides that have been modified to comprise a thiol group or comprise an amino group can be used.

In certain embodiments, at least one of the agent and the biomaterial is a member selected from the group consisting of an antibody, a viral vector, a growth factor, a bioactive polypeptide, a polynucleotide coding for the bioactive polypeptide, a cell regulatory small molecule, a peptide, a protein, an oligonucleotide, a gene therapy agent, a gene transfection vector, a receptor, a cell, a drug, a drug delivering agent, nitric oxide, an antimicrobial agent, an antibiotic, antimitotic, an antisecretory agent, an anti-cancer chemotherapeutic agent, dexamethasone, an extracellular matrix, free radical scavenger, iron chelator, an antioxidant, an imaging agent, and a radiotherapeutic agent. In certain embodiments, at least one of the agent and the biomaterial is an anti-knob antibody, an adenovirus, a D1 domain of the Coxsackie-adenovirus receptor (CAR D1), insulin, an angiogenic peptide, an antiangiogenic peptide, avidin, biotin, IgG, protein A, transferrin, and a receptor for transferrin.

Suitable biomaterials include pharmaceuticals, nucleic acid sequences, such as transposons, signaling proteins that facilitate wound healing, such as TGF-β, FGF, PDGF, IGF and GH proteins that regulate cell survival and apoptosis, such as Bcl-1 family members and caspases; tumor suppressor proteins, such as the retinoblastoma, p53, PAC, DCC. NF1, NF2, RET, VHL and WT-1 gene products; extracellular matrix proteins, such as laminins, fibronectins and integrins; cell adhesion molecules such as cadherins, N-CAMs, selectins and immunoglobulins; anti-inflammatory proteins such as Thymosin beta-4, IL-10 and IL-12.

In certain embodiments, the biomaterial includes at least one of heparin, covalent heparin, or another thrombin inhibitor, hirudin, hirulog, argatroban, D-phenylalanyl-L-poly-L-arginyl chloromethyl ketone, or another antithrombogenic agent, or mixtures thereof; urokinase, streptokinase, a tissue plasminogen activator, or another thrombolytic agent, or mixtures thereof; a fibrinolytic agent; a vasospasm inhibitor; a calcium channel blocker, a nitrate, nitric oxide, a nitric oxide promoter or another vasodilator; an antimicrobial agent or antibiotic; aspirin, ticlopidine, a glycoprotein IIb/IIIa inhibitor or another inhibitor of surface glycoprotein receptors, or another antiplatelet agent; colchicine or another antimitotic, or another microtubule inhibitor, dimethyl sulfoxide (DMSO), a retinoid or another antisecretory agent; cytochalasin or another actin inhibitor; a remodeling inhibitor; deoxyribonucleic acid, an antisense nucleotide or another agent for molecular genetic intervention; methotrexate or another antimetabolite or antiproliferative agent; tamoxifen citrate, Taxol™ or derivatives thereof, or other anti-cancer chemotherapeutic agents; dexamethasone, dexamethasone sodium phosphate, dexamethasone acetate or another dexamethasone derivative, or another anti-inflammatory steroid or non-steroidal anti-inflammatory agent; cyclosporin or another immunosuppressive agent; trapidal (a PDGF antagonist), angiogenin, angiopeptin (a growth hormone antagonist), a growth factor or an anti-growth factor antibody, or another growth factor antagonist; dopamine, bromocriptine mesylate, pergolide mesylate or another dopamine agonist; radiotherapeutic agent; iodine-containing compounds, barium-containing compounds, gold, tantalum, platinum, tungsten or another heavy metal functioning as a radiopaque agent; a peptide, a protein, an enzyme, an extracellular matrix component, a cellular component or another biologic agent; captopril, enalapril or another angiotensin converting enzyme (ACE) inhibitor; ascorbic acid, alpha tocopherol, superoxide dismutase, deferoxamine, a 21-amino steroid (lasaroid) or another free radical scavenger, iron chelator or antioxidant; a ¹⁴C-, ³H-, ³²P- or ³⁶S-radiolabelled form or other radiolabelled form of any of the foregoing; a hormone; estrogen or another sex hormone; AZT or other antipolymerases; acyclovir, famciclovir, rimantadine hydrochloride, ganciclovir sodium or other antiviral agents; 5-aminolevulinic acid, meta-tetrahydroxyphenylchlorin, hexadecafluoro zinc phthalocyanine, tetramethyl hematoporphyrin, rhodamine 123 or other photodynamic therapy agents; an IgG2 Kappa antibody against Pseudomonas aeruginosa exotoxin A and reactive with A431 epidermoid carcinoma cells, monoclonal antibody against the noradrenergic enzyme dopamine beta-hydroxylase conjugated to saporin or other antibody targeted therapy agents; gene therapy agents; and enalapril and other prodrugs, or a mixture of any of these.

Additionally, the biomaterial can be a component of any affinity-ligand pair. Examples of such affinity-ligand pairs include avidin-biotin and IgG-protein A. Furthermore, the biomaterial can be a component of any receptor-ligand pair. One example is transferrin and its receptor. Other affinity-ligand pairs include powerful hydrogen bonding or ionic bonding entities such as chemical complexes. Examples of the latter include metallo-amine complexes. Other such attractive complexes include nucleic acid base pairs via immobilizing oligonucleotides of a specific sequence, especially antisense. Nucleic acid decoys or synthetic analogues can also be used as pairing agents to bind a designed gene vector with attractive sites. Furthermore, DNA binding proteins can also be considered as specific affinity agents; these include such entities as histones, transcription factors, and receptors such as the gluco-corticoid receptor.

In one embodiment, the biomaterial is an anti-nucleic acid antibody. The antibody can therefore specifically bind a nucleic acid, which encodes a product (or the precursor of a product) that decreases cell proliferation or induces cell death, thereby mitigating the problem of restenosis in arteries and other vessels. The nucleic acid tethered to a support via the antibody can efficiently transfect/transduce cells. In general terms, the field of “gene therapy” involves delivering into target cells some polynucleotide, such as an antisense DNA or RNA, a ribozyme, a viral fragment, or a functionally active gene, that has a therapeutic or prophylactic effect on the cell or the organism containing it. The antibody can be a full-length (i.e., naturally occurring or formed by normal immuno-globulin gene fragment recombinatorial processes) immunoglobulin molecule (e.g., an IgG antibody, or IgM or any antibody subtype) or an immunologically active (i.e., specifically binding) portion of an immunoglobulin molecule. The antibody comprises one or more sites, which specifically bind with a nucleic acid (i.e., which does not substantially bind other types of molecules). The binding site can be one, which binds specifically with a nucleic acid of a desired type without regard to the nucleotide sequence of the nucleic acid. The binding site can, alternatively, be one which binds specifically only with a nucleic acid comprising a desired nucleotide sequence. Preferably, the antibody is a thiol-modified antibody.

The complex formed between a polynucleotide and a cognate antibody can be immobilized on a surface of the filaments of the invention such that, when the filaments are exposed to a physiological environment in situ, the attached polynucleotide is released, over time, in a manner that enhances delivery of the polynucleotide to cells in the proximity. Surprisingly, DNA transfer by way of immunospecific tethering maintains the nucleic acid in regions that are subject to gene therapy.

Examples of suitable antibodies include Fv, F(ab), and F(ab′)₂ fragments, which can be generated is conventional fashion, as by treating an antibody with pepsin or another proteolytic enzyme. The nucleic acid-binding antibody can be polyclonal antibody or a monoclonal antibody. A “monoclonal” antibody comprises only one type of antigen binding site that specifically binds with the nucleic acid. A “polyclonal” antibody can comprise multiple antigen binding sites that specifically bind the nucleic acid. An antibody employed in this invention preferably is a full-length antibody or a fragment of an antibody, such as F(ab′)₂, that possesses the desired binding properties.

A nucleic acid for use in the present invention can be any polynucleotide that one desires to transport to the interior of a cell. In this context, a “therapeutic polynucleotide” is a polymer of nucleotides that, when provided to or expressed in a cell, alleviates, inhibits, or prevents a disease or adverse condition, such as inflammation and/or promotes tissue healing and repair (e.g., wound healing). The nucleic acid can be composed of deoxyribonucleosides or ribonucleosides, and can have phosphodiester linkages or modified linkages, such as those described below. The phrase “nucleic acid” also encompasses polynucleotides composed of bases other than the five that are typical of biological systems: adenine, guanine, thymine, cytosine and uracil.

A suitable nucleic acid can be DNA or RNA, linear or circular and can be single- or -double-stranded. The “DNA” category in this regard includes cDNA; genomic DNA; triple helical, supercoiled, z-DNA and other forms of DNA; polynucleotide analogs; an expression construct that comprises a DNA segment coding for a protein, including a therapeutic protein; so-called “antisense” constructs that, upon transcription, yield a ribozyme or an antisense RNA; viral genome fragments, such as viral DNA; plasmids and cosmids; and a gene or gene fragment.

The nucleic acid also can be RNA, for example, antisense RNA, catalytic RNA, catalytic RNA/protein complex (i.e., a “ribozyme”), and expression construct comprised of RNA that can be translated directly, generating a protein, or that can be reverse transcribed and either transcribed or transcribed and then translated, generating an RNA or protein product, respectively; transcribable constructs comprising RNA that embodies the promoter/regulatory sequence(s) necessary for the generation of DNA by reverse transcription; viral RNA; and RNA that codes for a therapeutic protein, inter alia. A suitable nucleic acid can be selected on the basis of a known, anticipated, or expected biological activity that the nucleic acid will exhibit upon delivery to the interior of a target cell or its nucleus.

The length of the nucleic acid is not critical to the invention. Any number of base pairs up to the full-length gene may be transfected. For example, the nucleic acid can be linear or circular double-stranded DNA molecule having a length from about 100 to 10,000 base pairs in length, although both longer and shorter nucleic acids can be used.

The nucleic acid can be a biomaterial, such as an antisense DNA molecule that inhibits mRNA translation. Alternatively, the nucleic acid can encode a biomaterial, such as a transcription or translation product which, when expressed by a target cell to which the nucleic acid is delivered, has a therapeutic effect on the cell or on a host organism that includes the cell. Examples of therapeutic transcription products include proteins (e.g., antibodies, enzymes, receptors-binding ligands, wound-healing proteins, anti-restenotic proteins, anti-restenotic proteins, anti-oncogenic proteins, and transcriptional or translational regulatory proteins), antisense RNA molecules, ribozymes, viral genome fragments, and the like. The nucleic acid likewise can encode a product that functions as a marker for cells that have been transformed. Illustrative markers include proteins that have identifiable spectroscopic properties, such as green fluorescent protein (GFP) and proteins that are expressed on cell surfaces (i.e., can be detected by contacting the target cell with an agent which specifically binds the protein). Also, the nucleic acid can be a prophylactic agent useful in the prevention of disease.

A nucleic-acid category that is important to the present invention encompasses polynucleotides that encode proteins that affect wound-healing. For example, the genes egf, tgf, kgf, hb-egf, pdgf, igf, fgf-1, fgf-2, vegf, other growth factors and their receptors, play a considerable role in wound repair.

Another category of polynucleotides, coding for factors that modulate or counteract inflammatory processes, also is significant for the present invention. Also relevant are genes that encode an anti-inflammatory agent such as MSH, a cytokine such as IL-10, or a receptor antagonist that diminishes the inflammatory response.

Suitable polynucleotides can code for an expression product that induces cell death or, alternatively, promotes cell survival, depending on the nucleic acid. These polynucleotides are useful not only for treating tumorigenic and other abnormal cells but also for inducing apoptosis in normal cells. Accordingly, another notable nucleic-acid category for the present invention relates to polynucleotides that, upon expression, encode an anti-oncogenic protein or, upon transcription, yield an anti-oncogenic antisense oligonucleotide. In this context, the phrases “anti-oncogenic protein” and “anti-oncogenic antisense oligonucleotide” respectively denote a protein or an antisense oligonucleotide that, when provided to any region where cell death is desired, or the site of a cancerous or precancerous lesion in a subject, prevents, inhibits, reverses abnormal and normal cellular growth at the site or induces apoptosis of cells. Delivery of such a polynucleotide to cells, pursuant to the present invention, can inhibit cellular growth, differentiation, or migration to prevent movement or unwanted expansion of tissue at or near the site of transfer. Illustrative of this anti-oncogenic category are polynucleotides that code for one of the known anti-oncogenic proteins. Such a polynucleotide would include, for example, a nucleotide sequence taken or derived from one or more of the following genes: abl, akt2, apc, bcl2-alpha, bcl2-beta, bcl3, bcl3, bcl-x, bad, bcr, brcal, brca2, cbl, ccndl, cdk4, crk-II, csflr/fms, dbl, dcc, dpc4/smad4, e-cad, e2fl/rbap, egfr/erbb-1, elk1, elk3, eph, erg, ets1, ets2, fer, fgr/src2, fos, fps/fes, fral, fra2, fyn, hck, hek, her2/erbb-2/neu, her3/erbb-3, her4/erbb-4, hras1, hst2, hstfl, ink4a, ink4b, int2/fgf3, jun, junb, jund, kip2, kit, kras2a, kras2b, ck, lyn, mas, max, mcc, met, mlh1, mos, msh2, msh3, msh6, myb, myba, mybb, myc, mycl1, mycn, nfl, nf2, nras, p53, pdgfb, pim1, pms1, pms2, ptc, pten, raft, rb1, rel, ret, ros1, ski, src1, tal1, tgfbr2, thra1, thrb, tiam1, trk, vav, vhl, waf1, wnt1, wnt2, wt1 and yes1. By the same token, oligonucleotides that inhibit expression of one of these genes can be used as anti-oncogenic antisense oligonucleotides.

Nucleic acids having modified internucleoside linkages also can be used as biomaterial according to the present invention. For example, nucleic acids can be employed that contain modified internucleoside linkages, which exhibit increased nuclease stability. Such polynucleotides include, for example, those that contain one or more phosphonate, phosphorothioate, phosphorodithioate, phosphoramidate methoxyethyl phosphoramidate, formacetal, thioformacetal, diisopropylsilyl, acetamidate, carbamate, dimethylene-sulfide (—CH₂—S—CH₂—), dimethylene-sulfoxide (—CH₂—SO—CH₂—), dimethylenesulfone (—CH₂—SO₂—CH₂—), 2′-O-alkyl, and 2′-deoxy-2′-fluoro-phosphorothioate internucleoside linkages.

For present purposes, a nucleic acid can be prepared or isolated by any conventional means typically used to prepare or isolate nucleic acids. For example, DNA and RNA can be chemically synthesized using commercially available reagents and synthesizers by known methods. For example, see Gait, 1985, in: OLIGONUCLEOTIDE SYNTHESIS: A PRACTICAL APPROACH (IRL Press, Oxford, England). RNA molecules also can be produced in high yield via in vitro transcription techniques, using plasmids such as SP65, available from Promega Corporation (Madison, Wis.). The nucleic acid can be purified by any suitable means, and many such means are known. For example, the nucleic acid can be purified by reverse-phase or ion exchange HPLC, size exclusion chromatography, or gel electrophoresis. Of course, the skilled artisan will recognize that the method of purification will depend in part on the size of the DNA to be purified. The nucleic acid also can be prepared via any of the innumerable recombinant techniques that are known or that are developed hereafter.

A suitable nucleic acid can be engineered into a variety of known host vector systems that provide for replication of the nucleic acid on a scale suitable herein. Vector systems can be viral or non-viral. Particular examples of viral vector systems include adenovirus, retrovirus, adeno-associated virus and herpes simplex virus. Preferably, an adenovirus vector is used. A non-viral vector system includes a plasmid, a circular, double-stranded DNA molecule. Viral and nonviral vector systems can be designed, using known methods, to contain the elements necessary for directing transcription, translation, or both, of the nucleic acid in a cell to which is delivered. Methods, which are known to a skilled artisan can be used to construct expression constructs having the protein coding sequence operably linked with appropriate transcriptional/translational control signals. These methods include in vitro recombinant DNA techniques and synthetic techniques. For instance, see Sambrook et al., 1989, MOLECULAR CLONING: A LABORATORY MANUAL (Cold Spring Harbor Laboratory, New York).

A nucleic acid encoding one or more proteins of interest can be operatively associated with a variety of different promoter/regulator sequences. The promoter/regulator sequences can include a constitutive or inducible promoter, and can be used under the appropriate conditions to direct high level or regulated expression of the gene of interest. Particular examples of promoter/regulatory regions that can be used include the cytomegalovirus (CMV) promoter/regulatory region and the promoter/regulatory regions associated with the SV40 early genes or the SV40 late genes. Preferably, the human CMV promoter is used, but substantially any promoter/regulatory region directing high level or regulated expression of the gene of interest can be used.

It also is within the scope of the present invention that the employed nucleic acid contains a plurality of protein-coding regions, combined on a single genetic construct under control of one or more promoters. The two or more protein-coding regions can be under the transcriptional control of a single promoter, and the transcript of the nucleic acid can comprise one or more internal ribosome entry sites interposed between the protein-coding regions. Thus, a myriad of different genes and genetic constructs can be utilized.

Antibodies specific for non-viral vectors or nucleic acids may require use of a transfection agent to enhance administration of nucleic acids. The transfection agent is a cationic macromolecule that is positively charged, comprises two or more art-recognized modular units (e.g., amino acid residues, fatty acid moieties, or polymer repeating units), and is preferably capable of forming supermolecular structures (e.g., aggregates, liposomes or micelles) at high concentration in aqueous solution or suspension. Among the types of cationic macromolecules that can be used are cationic lipid and polycationic polypeptides.

The amount of the transfection agent to be used when transfecting cells can be calculated based on the nucleic acid content of the biomaterial. The capacity of the medium comprising or containing the transfection agent can also affect the amount of transfection agent to be used. Cells can be infected with viral vectors by methods known in the art.

The biomaterial can also be a drug. The term “drug” as used herein is defined a chemical capable of administration to an organism, which modifies or alters the organism's physiology and intended for use in the treatment or prevention of disease. Specific non-limiting examples of drugs which can be used in this invention include paclitaxel, docetaxel and derivatives, epothilones, nitric oxide release agents, heparin, aspirin, coumadin, PPACK, hirudin, polypeptide from angiostatin and endostatin, methotrexate, 5-fluorouracil, estradiol, P-selectin Glycoprotein ligand-1 chimera, abciximab, exochelin, eleutherobin and sarcodictyin, fludarabine, sirolimus, tranilast, VEGF, transforming growth factor (TGF)-beta, insulin-like growth factor (IGF), platelet derived growth factor (PDGF), fibroblast growth factor (FGF), and RGD peptide.

Also provided is a method for delivery of a biomaterial to a cell, the method comprising (1) providing the device of the invention having a monomolecular layer of a water soluble photo-activatable polymer covalently attached to the at least one of the filaments, (2) providing a biomaterial having a plurality of active groups, wherein the biomaterial is covalently attached to the monomolecular layer, and (3) administering the device to the cell.

In certain embodiments of the method for delivery of a biomaterial to a cell, the method comprises providing the device of the invention having the biomaterial in communication with the at least one of the filaments and administering the device to the cell. Non-limiting examples of communication of the biomaterial and the filament is (1) by mixing the biomaterial with a polymer or polymers of the filament prior to making the filament and (2) by associating the biomaterial with a fiber's polymer followed by application (i.e., via electrospinning) onto the filament. The device can then be used to deliver the biomaterial to the interior of a cell or a tissue in need of, for example, gene therapy.

A Stent Delivery System

Also provided is a stent delivery system, comprising a delivery vessel, a stent, wherein the stent is placed in the delivery vessel in the collapsed state, and a delivery unit capable of delivering the stent from the delivery vessel into a body lumen, wherein the stent is allowed to expand from the collapsed state to an expanded state such that an expanded transverse diameter of the stent is substantially identical to a manufactured transverse diameter of the stent and thereby a manufactured shape of the stent is retained. In certain embodiments of the stent delivery system, the collapsed state is achieved by stretching the stent in a longitudinal direction. The stent itself is described above. In the preferred embodiment, the stent's filaments are in association with the water-soluble photo-activatable polymer via irradiation and biopolymer, which is covalently attached to the water-soluble photo-activatable polymer, wherein the biomaterial is at least one of an anti-knob antibody, an adenovirus, a D1 domain of the Coxsackie-adenovirus receptor, insulin, an angiogenic peptide, and an antiangiogenic peptide.

Further provided is a process of manufacturing of the stent delivery system described above, the process comprising (1) providing the stent, (2) providing the delivery vessel, (3) providing the delivery unit; and (4) installing the stent into the delivery vessel.

Also provided is a process for delivery of a stent to a body lumen, the process comprising providing the stent delivery system, providing the body lumen, contacting the delivery vessel with the body lumen, deploying the stent, wherein the stent is allowed to expand from the collapsed state to the expanded state such that the expanded transverse diameter of the stent is substantially identical to the manufactured transverse diameter of the stent and thereby the manufactured shape of the stent is retained.

Typically, the stents are delivered intraluminally through a percutaneous incision through the femoral or renal arteries. A stent is mounted on the distal end of an elongated catheter, typically on the balloon portion of a catheter, and the catheter and stent are advanced intraluminally to the site where the stent is to be implanted. Typically with expandable stents, the balloon portion of the catheter is inflated to expand the stent radially outwardly into contact with the arterial wall, whereupon the stent undergoes plastic deformation and remains in an expanded state to hold open and support the artery.

With respect to self-expanding stents, typically a retractably sheath is positioned over the self-expanding stent which is mounted on the distal end of the catheter. Once the catheter has been advanced intraluminally to the site where the stent is to be implanted, the sheath is withdrawn thereby allowing the self-expanding stent to expand radially outwardly into contact with the arterial wall, thereby holding open and supporting the artery.

The invention will be illustrated in more detail with reference to the following Examples, but it should be understood that the present invention is not deemed to be limited thereto.

### EXAMPLES

### Example 1

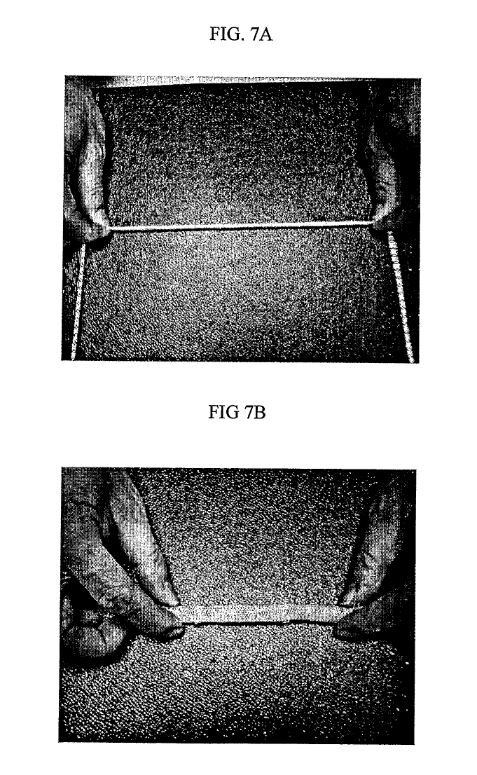

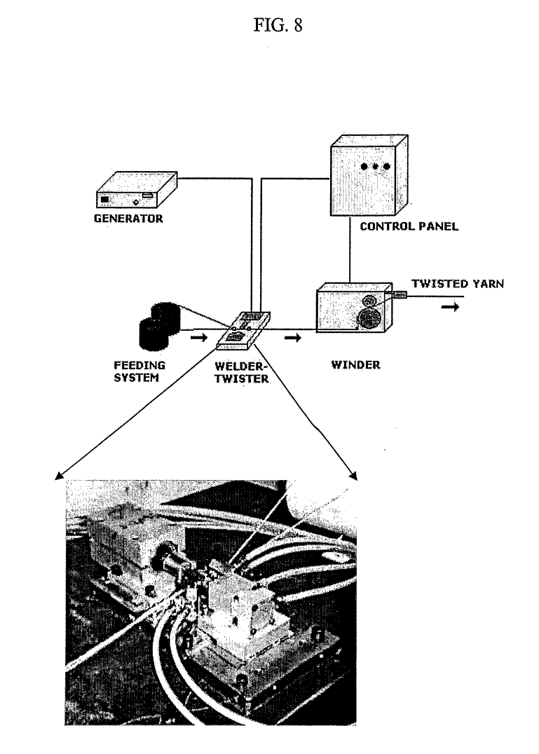

A braided stent made of polyester monofilaments functionalized by coupling with nanofibers was prepared. A 24-carrier braider was used with a 450° braiding angle. The braided stent on a mandrel was subjected to a pressure at 0.1 MPa to create impression points or grooves and heat set at 150° C. for one hour and cooled to room temperature. FIG. 7A shows the sent in a deployed or extended position. FIG. 7B shows a polymer stent in a stretched position prior to its placement into a delivery vessel.

### Example 2

Bioactive monofilament is prepared by the ultrasonic welding of false twisted yarn (FIG. 8) wherein the nanofiber DNA fibers are fed/wrapped onto the monofilament and thermomechanically bonded together to form an integral linear assembly. The bioactive monofilament can simultaneously serve as a braiding yarn and biomaterial.

### Example 3

Placement of nanofiber on expanded stent is shown in FIGS. 9A and 9B. FIG. 9A is showing brading of the stent over a mandrel. The biomaterial in a form of a nanofiber was electrospinned on a rotating braided stent from a spinneret (see FIG. 9B). The nanofiber/monofilament assembly was heat set in the deployed (expanded) form. The biomaterial is DNA.

### Example 4

In this example, the bioactive function is conferred to the stent by treating either the stent or filaments prior to braiding with water-soluble photo-activatable polymers such as photo-active poly(allylamine) benzophenone (PAA-BzPh) or poly(allylamine)-based benzophenone further modified to contain 2-pyridyldithio groups (PDT-BzPh).

### Synthesis of PAA-BzPh

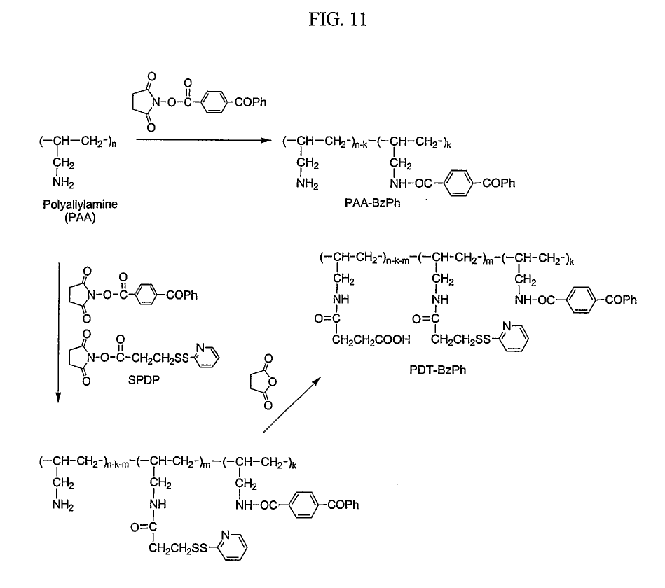

Synthesis of PAA-BzPh is demonstrated in FIG. 11. Poly(allylamine) (PAA) base was prepared from PAA hydrochloride (Sigma-Aldrich, St. Louis, Mo.; MW=70 KDa) by treatment in aqueous medium with a strong anionite Dowex G-55 followed by replacement of water by 2-propanol. A 5.1% solution of PAAbase in 2-propanol (4.06 g, containing 3.65 mmol of amino groups) was diluted with CH₂Cl₂ (7 ml) and cooled on an ice bath. Succinoyl 4-benzoylbenzoate (Sigma-Aldrich, 236 mg, 0.73 mmol) in CH₂Cl₂ (12 ml) was added over a 10-min period. The mixture was stirred near 0° C. for 10 min, then warmed to room temperature and acidified with concentrated HCl (0.24 ml, 2.9 mmol). The resulting suspension was dried in vacuo, resuspended in CH₂Cl₂, and the precipitate was filtered off. After washing with CH₂Cl₂ and pentane, 0.544 g of PAA-BzPh hydrochloride were obtained. A ¹H NMR study of this polymer (utilizing D₂O) indicated that 20% of polymer's amino groups were modified with 4-benzoylbenzoic residues (broad signal at 6.9-8.0 ppm). Analogously, using the calculated amount of fluorescein isothiocyanate (FITC) (Sigma-Aldrich, St. Louis, Mo.,) simultaneously with succinoyl 4-benzoylbenzoate in the reaction with PAA base, FITC-labeled PAA-BzPh having about 20% of 4-benzoylbenzoic residues and about 2% of the FITC label was prepared.

### Synthesis of PDT-BzPh

Synthesis of PDT-BzPh, the water-soluble photo-activatable polymer, is shown in FIG. 11. A 5.1% solution of PAA base in 2-propanol (2.671 g, containing 2.40 mmol of amino groups) was diluted with CH₂Cl₂ (5 ml) and cooled on ice. Succinoyl 4-benzoylbenzoate (145 mg, 0.45 mmol) and SPDP (Pierce Biotechnology Inc, Rockford, Ill., 281 mg, 0.90 mmol) were simultaneously dissolved in CH₂Cl₂ (8 ml) and introduced over a 5-min period. The mixture was stirred near 0° C. for 15 min, and succinic anhydride (130 mg, 1.30 mmol) was added at once. The stirring at 0° C. was continued for 0.5 h, the mixture was dried in vacuo and extracted first with ethyl acetate and then with water. The polymeric residue was dissolved in water (15 ml) with addition of KHCO₃ (0.3 g, 3.0 mmol). The solution was filtered and acidified with H₃PO₄ to pH of 3.5. The precipitate was filtered off, washed with water, and air-dried PDT-BzPh (488 mg) was obtained. A ¹H NMR study of this polymer (utilizing D₂O and K₂CO₃ at pH 9) indicated that about 40% of 2-pyridyldithio groups and about 20% of 4-benzoylbenzoic residues were attached to the PAA backbone. The rest of amino groups was modified with 3-carboxypropionyl residues resulting from succinic anhydride.

### Example 5

### Photo-Immobilization of Polymeric Modifiers onto Matrix

Modification of Polymeric Matrix with PAA-BzPh.

An aqueous solution (2 mg/ml) of PAA-BzPh or its FITC-labeled variant was mixed with an equal volume of a buffer containing 0.1M NH₄OAc and 0.05M NH₃. PU Tecothane TT-1074A films or polyester (PE) fibers were immersed into the mixture for 5-60 min, rinsed with a 1% solution of NH₃ and dried on a filter paper. The polymers were irradiated under an UV-lamp (UVGL-25, long wave) for 15-30 min to achieve the covalent binding of modifiers to the polymer surface. Finally, the surface modified polymers were thoroughly washed with diluted (2%) HCl and water.

Modification of Polymeric Matrix with PDT-BzPh.

PDT-BzPh (30 mg) was dissolved in water (30 ml) by addition of KHCO₃ (20 mg) and acidified with a 20% solution of KH₂PO₄ (1 ml). PU films and PE fibers were soaked in the resulting mixture for 5-40 min., rinsed with 0.1% acetic acid, dried and irradiated as above. Finally, the polymers were exhaustively washed with 0.1M KHCO₃ and water.

### Example 6

### Fluorescent Labeled PAA-BzPh Studies Demonstrating Attachment of Biomolecules

Fluorescence microscopy of PU films and PE fibers surface modified with FITC-labeled PAA-BzPh was performed and confirmed the presence of the modifier bound to the polymer surfaces, wherein. As controls, samples with non-modified polymers were used. The samples with modified polymers displayed green color in fluorescence microscopy.

### Example 7

### Cell Culture Data Demonstrating Antibody Linkage of Cy3 labeled GFP-Adenovirus

PU Matrix

Surface-aminated PU films (group NH₂-A) were reacted with LC-sulfo-SPDP dissolved in PBS (9 mg/ml; 1 ml; 90 min). Then, the films were extensively washed in PBS and reacted in 5% BSA with anti-knob Ab (0.66 mg/ml) reduced with 1.5 mg of 2-mercaptoethylamine for 90 min at 37° C. Prior to conjugation, Ab was purified by gel filtration using a desalting column equilibrated with degassed PBS containing 10 mM EDTA. The conjugation was allowed to run for 38 hours at room temperature (RT) under mild shaking. Next, the films were washed in PBSx3 and immersed in the suspension of 10¹¹ particles of Cy3-labeled adenovirus (Cy3-AdV-GFP) in 1.5 ml of 5% BSA/PBS. Surface aminated films that were not modified by antiknob Ab (NH₂—B) served as controls. Immunoconjugation was carried out for 12 hours at RT under mild shaking. Finally, the films were washed in PBS and examined under fluorescent microscope to assess tethering of Cy3-labeled adenoviruses. A uniform virus coverage of the surface was observed for the films conjugated with antiknob Ab, while the control films were virtually non-fluorescent.

PDT-BzPh-modified PU Tecothane TT-1074A films were directly modified with the reduced antiknob Ab. After washing the films (PDT-A) along with the control samples, the PDT-BzPh-modified PU samples that were not conjugated with the antilnob Ab (PDT-B), both PDT-A and PDT-B were incubated with Cy3-labeled GFP-AdV. Antibody reduction, purification and conjugation, and virus tethering were carried out according the procedures outlined above. Similar to the results obtained for the surface-aminated PU samples, a uniform fluorescent AdV layer was observed for the Ab-mediated AdV tethering, while no Cy-3-labeled AdV was bound to the surface of control films.

PE Matrix

PDT-BzPh-modified PE fibers were reacted with 2 mg of antiknob antibody reduced with 10 mg of 2-mercaptoethylamine at 37° C. for 1 hour. Prior to utilization, the reduced Ab was purified using a desalting column equilibrated with degassed PBS/10 mM EDTA. The conjugation was carried out in 5% BSA/PBS for 20 hours at room temperature under mild shaking.

After PBS×3 washing, the Ab-coupled fibers were immersed into the suspension of 5×10¹¹ particles of Cy3-labeled adenovirus in 1.5 ml of 5% BSA/PBS. Immune conjugation was carried out for 14 hours at RT under mild shaking. Immobilization of Cy3-AdV on the surface of PDT-BzPh-modified PE fibers was confirmed by fluorescent microscopy.

While the invention has been described in detail and with reference to specific examples thereof, it will be apparent to one skilled in the art that various changes and modifications can be made therein without departing from the spirit and scope thereof.

1. A device comprising polymeric filaments, wherein at least one of the filaments includes at least one groove for slidably retaining at least one other filament, such that the device is adapted to revert to a tubular lattice structure when allowed to expand from a collapsed state.

2. The device of claim 1, wherein the tubular lattice structure has an expanded transverse diameter substantially identical to a manufactured transverse diameter.

3. The device of claim 1, wherein the manufactured transverse diameter is about 2 mm to about 300 mm.

4. The device of claim 1, wherein the manufactured transverse diameter is heat set at a temperature equal to or above a glass transition temperature of the filaments.

5. The device of claim 4, wherein the temperature is from about 100° C. to about 200° C.

6. The device of claim 1, wherein the filaments have a diameter from about 10 micron to about 1000 micron.

7. The device of claim 1, wherein at least three of the polymeric filaments are braided at a braiding angle of about 5° to about 85°.

8. The device of claim 7, wherein the braiding angle is from 40° to 50°.

9. The device of claim 1, wherein the at least one groove has a combination of a depth and a height suitable to inhibit a movement of adjacent filaments in a transverse direction beyond the manufactured transverse diameter without inhibiting the movement in a longitudinal direction.

10. The device of claim 1, wherein the at least one of said filaments further comprises a plurality of grooves.

11. The device of claim 1, wherein the polymeric filaments comprise a thermoplastic polymer, wherein the polymer is selected from a group consisting of poly(ester), poly(lactic acid), poly(glycolic acid), poly(lactide-co-glycolide), poly(caprolactone), mixtures and copolymers thereof.

12. The device of claim 1, wherein the polymeric filaments are at least one of monofilaments or multifilaments.

13. The device of claim 12, wherein the polymeric filaments further comprise at least one of a biomaterial and an agent having a reactive group, wherein the reactive group is adapted to covalently react with a biomaterial.

14. The device of claim 13, wherein the reactive group is a member selected from the group consisting of an amino group, a thiol-reactive group, a carboxy group, a thiol group, a protected thiol group, an acyl hydrazine group, an epoxy group, an aldehyde group, and a hydroxy group.

15. The device of claim 14, wherein the thiol-reactive group is a member selected from the group consisting of a 2-pyridyldithio group, a 3-carboxy-4-nitrophenyldithio group, a maleimide group, an iodoacetamide group, and a vinylsulfonyl.

16. The device of claim 13, wherein at least one of the agent and the biomaterial is a member selected from the group consisting of an antibody, a viral vector, a growth factor, a bioactive polypeptide, a polynucleotide coding for the bioactive polypeptide, a cell regulatory small molecule, a peptide, a protein, an oligonucleotide, a gene therapy agent, a gene transfection vector, a receptor, a cell, a drug, a drug delivering agent, nitric oxide, an antimicrobial agent, an antibiotic, antimitotic, an antisecretory agent, an anti-cancer chemotherapeutic agent, dexamethasone, an extracellular matrix, free radical scavenger, iron chelator, an antioxidant, an imaging agent, and a radiotherapeutic agent.

17. The device of claim 16, wherein at least one of the agent and the biomaterial is at least one of an anti-knob antibody, an adenovirus, a D1 domain of the Coxsackie-adenovirus receptor, insulin, an angiogenic peptide, and an antiangiogenic peptide.

18. The device of claim 13, wherein the agent is covalently attached to the at least one of the filaments.

19. The device of claim 18, wherein the agent is a water-soluble photo-activatable polymer comprising:
(a) a photo-activatable group, wherein the photo-activatable group is adapted to be activated by an irradiation source and to form a covalent bond between the water-soluble photo-activatable polymer and a surface having at least one carbon;
(b) a reactive group, wherein the reactive group is adapted to covalently react with the biomaterial;
(c) a hydrophilic group, wherein the hydrophilic group is present in an amount sufficient to make the water-soluble photo-activatable polymer soluble in water; and
(d) a polymer precursor.

20. The device of claim 19, wherein the water-soluble photo-activatable polymer is represented by a formula: 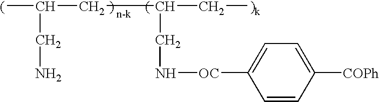
wherein n is 50 to 2000 and k is 10 to 1000.

21. The device of claim 19, wherein the water-soluble photo-activatable polymer is represented by a formula: 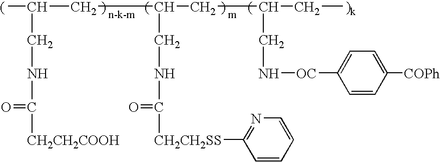
wherein n is 50 to 2000, k is 10 to 1000, and m is 10 to 1000.

22. The device of claim 13, further comprising a fiber in communication with the at least one of the filaments and, wherein the fiber is also in communication with the agent and/or the biomaterial, and wherein the fiber has a diameter of about 5 nm to about 10 microns.

23. The device of claim 22, wherein the agent and/or the biomaterial is applied to the fiber by coating, painting, stamping, printing, and/or spraying.

24. The device of claim 22, wherein the agent and/or the biomaterial is covalently attached to the fiber.

25. The device of claim 24, wherein the agent comprises water-soluble photo-activatable polymer and the biomaterial comprises an anti-knob antibody, an adenovirus, a D1 domain of the Coxsackie-adenovirus receptor, insulin, an angiogenic peptide, or an antiangiogenic peptide.

26. The device of claim 22, wherein the fiber consists essentially of the agent and/or the biomaterial.

27. The device of claim 1, wherein the tubular lattice structure is a stent.

28. A device comprising polymeric filaments, wherein at least one of said filaments includes at least one groove for slidably retaining at least one other filament, such that said device is adapted to expand from a collapsed state to form a tubular lattice structure having an expanded transverse diameter substantially identical to a manufactured transverse diameter.

29. A process of manufacturing the device of claim 1, the process comprising:
providing polymeric filaments;
arranging the polymeric filaments over a mandrel to form a tubular lattice assembly having interlacing junctions, wherein the mandrel has the manufactured transverse diameter;
heating the tubular lattice assembly at a temperature at least 10° above a glass transition temperature of the polymeric filaments; and
indenting at least one of the polymeric filaments at one or more of the interlacing junctions to make the at least one groove on the at least one of the polymeric filaments for slidably retaining the at least one other polymeric filament and thereby forming the tubular lattice structure.

30. The process of claim 29, further comprising contacting the at least one of the filaments with an agent having a reactive group, wherein the reactive group is adapted to covalently react with a biomaterial.

31. The process of claim 30, further comprising covalently attaching the agent to the at least one of the filaments.

32. The process of claim 30, further comprising contacting the at least one of the filaments with a fiber associated with the agent.

33. The process of claim 32, wherein the fiber has a diameter of about 5 nm to about 10 microns.

34. The process of claim 32, wherein contacting is done by at least one of an ultrasonic welding or an electrospinning process.

35. The process of claim 32, further comprising covalently attaching the agent to the at least one of the filaments.

36. The process of claim 29, wherein the temperature is from about 100° C. to about 200° C. and the heating is conducted for at least 30 minutes.

37. The process of claim 36, wherein the temperature is from 100° C. to 15° C.

38. The process of claim 30, wherein affecting the interlacing junctions is done by a press.

39. A stent delivery system, comprising:
a delivery vessel;
the device of claim 1 as a stent, wherein the stent is placed in the delivery vessel in the collapsed state; and
a delivery unit capable of delivering the stent from the delivery vessel into a body lumen, wherein the stent is allowed to expand from the collapsed state to an expanded state such that an expanded transverse diameter of the stent is substantially identical to a manufactured transverse diameter of the stent and thereby a manufactured shape of the stent is retained.

40. The stent delivery system of claim 39, wherein the collapsed state is achieved by stretching the stent in a longitudinal direction.

41. A process of manufacturing of the stent delivery system of claim 39, the process comprising:
providing the stent;
providing the delivery vessel;
providing the delivery unit; and
installing the stent into the delivery vessel and thereby forming the stent delivery system.

42. A process for delivery of a stent to a body lumen, the process comprising:
providing the stent delivery system of claim 39;
providing the body lumen;
contacting the delivery vessel with the body lumen; and
deploying the stent, wherein the stent is allowed to expand from the collapsed state to the expanded state such that the expanded transverse diameter of the stent is substantially identical to the manufactured transverse diameter of the stent and thereby the manufactured shape of the stent is retained and thereby delivering the stent.

43. A method for delivery of a biomaterial to a cell, the method comprising:
providing the device of claim 1 having a monomolecular layer of a water soluble photo-activatable polymer covalently attached to the at least one of the filaments;
providing a biomaterial having a plurality of active groups, wherein the biomaterial is covalently attached to the monomolecular layer; and
administering the device to the cell and thereby delivering the biomaterial.

44. The method of claim 43, wherein the biomaterial is a member selected from the group consisting of an antibody, a viral vector, a growth factor, a bioactive polypeptide, a polynucleotide coding for the bioactive polypeptide, a cell regulatory small molecule, a peptide, a protein, an oligonucleotide, a gene therapy agent, a gene transfection vector, a receptor, a cell, a drug, a drug delivering agent, nitric oxide, an antimicrobial agent, an antibiotic, an antimitotic, dimethyl sulfoxide, an antisecretory agent, an anti-cancer chemotherapeutic agent, steroidal and non-steroidal anti-inflammatories, hormones, an extracellular matrix, a free radical scavenger, an iron chelator, an antioxidant, an imaging agent, and a radiotherapeutic agent.

45. The method of claim 44, wherein the biomaterial is a member selected from the group consisting of an anti-knob antibody, an adenovirus, a D1 domain of the Coxsackie-adenovirus receptor, insulin, an angiogenic peptide, an antiangiogenic peptide, avidin, biotin, IgG, protein A, transferrin, and a receptor for transferrin.

46. A method for delivery of a biomaterial to a cell, the method comprising:
providing the device of claim 1 having the biomaterial in communication with the at least one of the filaments; and
administering the device to the cell and thereby delivering the biomaterial.

47. The method of claim 46, wherein the biomaterial is covalently attached to the filament by the water-soluble photo-activatable polymer.

48. The method of claim 46, wherein the biomaterial is a member selected from the group consisting of an antibody, a viral vector, a growth factor, a bioactive polypeptide, a polynucleotide coding for the bioactive polypeptide, a cell regulatory small molecule, a peptide, a protein, an oligonucleotide, a gene therapy agent, a gene transfection vector, a receptor, a cell, a drug, a drug delivering agent, nitric oxide, an antimicrobial agent, an antibiotic, an antimitotic, dimethyl sulfoxide, an antisecretory agent, an anti-cancer chemotherapeutic agent, steroidal and non-steroidal anti-inflammatories, hormones, an extracellular matrix, a free radical scavenger, an iron chelator, an antioxidant, an imaging agent, and a radiotherapeutic agent.
